(19) United States
(12) Patent Application Publication
(10) Pub. No.: US 2017/0290950 A1
(43) Pub. Date: Oct. 12, 2017

(54) SYNTHESIS AND USE OF POLY(GLYCEROL-SEBACATE) FILMS IN FIBROBLAST GROWTH REGULATION

(71) Applicant: WAKE FOREST UNIVERSITY HEALTH SCIENCES, Winston-Salem, NC (US)

(72) Inventors: William D. WAGNER, Clemmons, NC (US); Victoria Shuangbai JIANG, Alpharetta, GA (US)

(21) Appl. No.: 15/517,297

(22) PCT Filed: Oct. 7, 2015
(86) PCT No.: PCT/US15/54484

§ 371 Date: Apr. 6, 2017

Related U.S. Application Data
(60) Provisional application No. 62/061,416, filed on Oct. 8, 2014.

(51) Int. Cl. A61L 26/00; C08J 3/24; C08G 63/91; C08G 63/16

(52) U.S. Cl. CPC A61L 26/0019; C08G 63/16; C08J 3/24; A61L 26/0085; C08G 63/916; C08J 2367/02

(57) ABSTRACT

Wound repair materials and methods of using the same to inhibit excess fibrosis are disclosed.

### CROSS-REFERENCE TO RELATED APPLICATIONS

This international application claims the benefit of U.S. Provisional Application No. 62/061,416, filed Oct. 8, 2014, the entirety of which is incorporated herein by reference.

### FIELD OF THE INVENTION

The present invention relates generally to wound repair materials and more particularly, but not exclusively, to wound repair materials that inhibit excess fibrillogenesis or fibrosis.

### BACKGROUND OF THE INVENTION

During the proliferative phase of wound healing, fibroblast activation results in collagen deposition and the formation of a provisional extracellular matrix. The deposition of collagen at the wound and contraction of the wound during wound healing minimizes wound surface area, forms a tough and elastic barrier, and protects against bacterial infection and fluid loss.

In pathologic wound healing, however, fibroblasts deposit excess collagen into the matrix that inhibits wound healing and may result in excessive fibrosis, adhesions, and excessive scar formation.

Therefore, a need exists in the field for providing new materials that may be used during wound treatment and repair to inhibit excess fibrosis and reduce scar formation.

### SUMMARY OF THE INVENTION

The present invention meets the needs in the field for materials and methods that inhibit excess fibrosis and reduce scar formation by providing wound repair materials and methods of use thereof. More specifically, the invention provides bioabsorbable wound repair materials for inhibiting fibrosis and collagen fibrillogenesis at a wound. As used herein, a “bioabsorbable,” “bioresorbable,” “bioincorporable,” or “biodegradable” material is a material that may dissolve in the tissue or which may be incorporated or absorbed in the tissue as a substantially indistinguishable component.

In a first aspect, the invention provides a wound repair material for inhibiting fibrosis in wound tissue that includes a polymeric material, wherein the polymeric material may include a poly (glycerol sebacate) (PGS). The wound repair material may be porous or non-porous. Moreover, in certain embodiments, the polymeric material may include a derivative of PGS, such as poly (glycerol sebacate) acrylate (PGSA). In other embodiments, the polymeric material may be a pre-polymer or a non-cross-linked polymer that may be cross-linked in situ. Furthermore, the wound repair material of the invention may be a film, a sheet, a strip, a solution, or a suspension.

In an additional aspect, the invention provides for a repair material that may inhibit fibrosis and reduce scar formation at a wound. The repair material may include a prepolymeric material that may comprise PGS. Moreover, the repair material may be configured to provide a porous film that may increase oxygen availability at the wound.

In another aspect, the invention provides a method of inhibiting fibrosis in a wound in a patient in need thereof. The method may include applying a wound repair material to the wound where the wound repair material includes a polymeric material that may include poly (glycerol sebacate) (PGS). In one embodiment, the method includes delivering the polymeric material of the wound repair material as a pre-polymer. Furthermore, the method of the invention may include delivering the wound repair material and then cross-linking the polymeric material in situ by applying UV light.

In a still further aspect, the invention includes a method of manufacturing a wound repair material, where the wound repair material inhibits fibrosis in wound tissue and includes a polymeric material comprising PGS or a derivative thereof.

### BRIEF DESCRIPTION OF THE DRAWINGS

The foregoing summary and the following detailed description of the exemplary embodiments of the present invention may be further understood when read in conjunction with the appended drawings, in which:

FIG. 1 schematically illustrates the preparation of poly (glycerol sebacate) (PGS) from glycerol and sebacic acid.

FIG. 2 schematically illustrates the general steps of preparing porous and non-porous PGS films where: image 1 provides the PGS prepolymer before use; images 2 describe prepolymer plating and pore generation steps; and images 3 illustrate porous and non-porous PGS films after polymerization.

FIG. 3 graphically illustrates an MTS assay where 3T3 fibroblasts were seeded above non-porous (A) and porous (B) PGS films with 3T3 fibroblasts provided as a control group (C) seeded on plastic.

FIG. 4 graphically illustrates an MTS assay where 3T3 fibroblasts were seeded below non-porous (A) and porous (B) PGS films with 3T3 fibroblasts provided as a control group (C) seeded on plastic.

FIGS. 5A-5F pictorially illustrate confocal microscopy images that demonstrate the cell morphology, size, density, and character of 3T3 fibroblasts plated on or below PGS films. FIGS. 5A and 5B illustrate the cell morphology and size of 3T3 cells plated below non-porous PGS (FIG. 5A) and porous PGS (FIG. 5B) at 80× magnification after 3 days. FIGS. 5C and 5D illustrate cell density and character of 3T3 cells plated below porous PGS (FIG. 5C) and non-porous PGS (FIG. 5D) at 10× magnification after 3 days. FIGS. 5E and 5F illustrate cell density and character of 3T3 cells in the control group after 3 days (FIG. 5E) and 5 days (FIG. 5F) at 20× magnification.

### DETAILED DESCRIPTION OF THE INVENTION

The disclosed bioabsorbable polymer films and other wound repair materials of the invention can be used to control fibroblast proliferation and collagen deposition at a wound. In pathologic wound healing, fibroblasts deposit excess collagen into the matrix that inhibits wound healing and may result in excessive fibrosis, adhesions, and excessive scar formation.

The process of wound healing consists of three main stages: inflammation, tissue proliferation, and tissue remodeling. During the proliferative phase, fibroblast activation results in collagen deposition and the formation of a provisional extracellular matrix (ECM). Collagen deposition and contraction minimizes wound surface area and forms a tough, elastic barrier to protect against bacterial infection and fluid loss during wound remodeling and repair. In pathologic wound healing, fibroblasts deposit excess collagen into the matrix which can inhibit the wound healing process and result in keloid scar formation. Collagen deposition, in this case, needs to be regulated in both the fiber size and amount of fiber deposited to prevent excessive scarring.

Accordingly, the materials of the invention may include certain polymeric materials that control or inhibit fibroblast proliferation and collagen deposition to allow for the management of wounds by reducing both the formation of scar tissue and the contraction of the wound. Indeed, the present invention provides for bioabsorbable or biodegradable polymeric materials that can be used to control fibroblast proliferation and collagen deposition at the wound.

Considering the invention more broadly, it includes a wound repair material that inhibits fibrosis or excessive fibrillogenesis in or at the wound tissue. Wounds of the invention may be non-penetrating wounds (i.e., soft tissue damage that does not penetrate the full thickness of the skin), penetrating wounds (i.e., soft tissue damage that does penetrate the full thickness of the skin, such as surgical cuts), thermal wounds (i.e., wounds to soft tissue caused by heat), chemical wounds (i.e., wounds to soft tissue caused by contact with chemical agents), and/or electrical wounds (i.e., wounds to soft tissue caused by contact with electricity). However, the materials of the invention may be applied to all wounds that result in fibrosis and/or collagen fibrillogenesis.

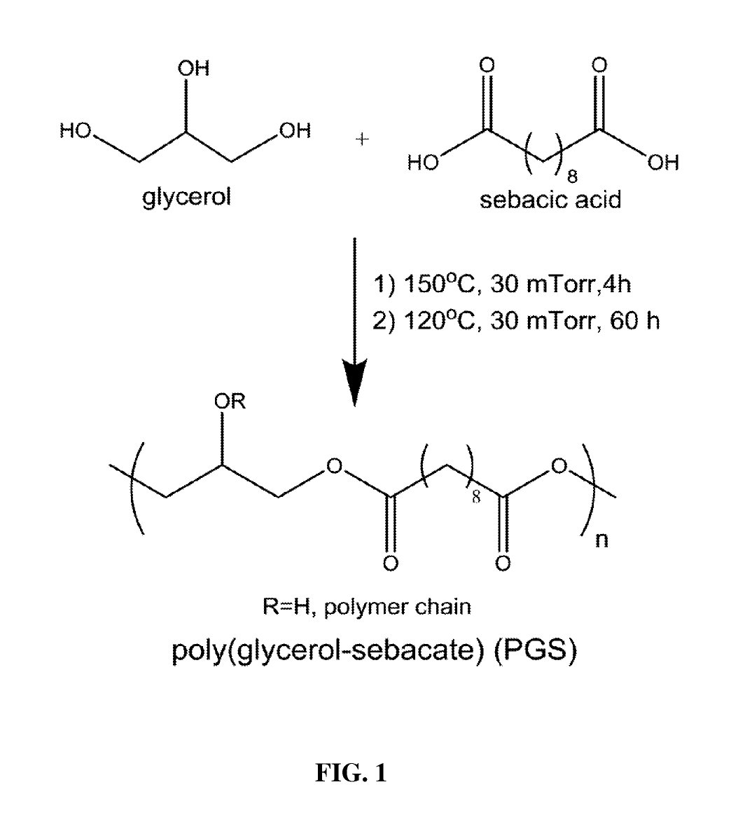

The wound repair materials of the invention may include polymeric materials with such polymeric materials comprising poly (glycerol sebacate) or a derivative thereof. Poly (glycerol-sebacate) (PGS) is a biopolymer that is rubbery, elastomeric in character, and similar to elastin. Moreover, PGS supports endothelial cell growth in vitro and has superior hemocompatibility in vitro. Furthermore, PGS is an FDA approved material and may biodegrade into non-toxic metabolites. A PGS prepolymer chemical core consists of glycerol, a fatty acid building block, and sebacate, a natural metabolite of ω-oxidation. PGS may be prepared from the condensation of glycerol and sebacic acid (FIG. 1). Previous studies have shown that PGS can support endothelial cell growth while maintaining superior hemocompatibility in vitro. This FDA approved material's non-toxic qualities, ability to biodegrade into non-toxic metabolites, and elastic character make it an ideal material for serving as a protective film for cutaneous injuries. As described herein, fibroblasts cultured in the presence of PGS films showed significant inhibition of fibroblast proliferation as compared to no material.

Polymeric materials of the invention include polymers. “Polymer,” as used herein, may include materials or compounds that are products of polymerization and is inclusive of prepolymers, homopolymers, copolymers, and terpolymers, for example. As used herein, the term “homopolymer” refers to a polymer resulting from the polymerization of a single monomer, i.e., a polymer consisting essentially of a single type of repeating unit. The term “copolymer” refers to polymers formed by the polymerization reaction of at least two different monomers and, moreover, the term copolymer is inclusive of random copolymers, block copolymers, and graft copolymers, for example. As used herein, the term “prepolymer,” may refer to partially polymerized monomers and represents an intermediate that may be fully polymerized upon curing. For example, as provided herein, a PGS pre-polymer may be synthesized from glycerol and sebacic acid. A PGS polymer film may then be prepared after curing the PGS pre-polymer at 120° C. for 60 hours under vacuum.

In addition to PGS, the polymeric material of the invention may include PGS derivatives such as, for example, poly (glycerol sebacate) acrylate (PGSA). Moreover, the polymeric material of the invention may include poly (ethylene glycol) diacrylate (PEG-DA). For example, the polymeric material of the invention may include a co-polymer of PGS and PEG-DA.

Polymers encompassed within a polymeric material may be cross-linked or non-cross-linked. In certain embodiments, the polymers of the polymeric material may be cross-linked by heating and/or the application of UV light. In other embodiments, the polymeric material and/or the wound repair material may include a light activated material that aids, enhances, or promotes the cross-linking of polymers of the polymeric material. The light activated material may include 2,2-dimethoxy-2-phenylacetophenone.

Furthermore, the wound repair materials of the invention may take a variety of forms to suit the needs of the person having ordinary skill in the art. The wound repair material may be a film, a sheet, a strip, a solution, or a suspension. For example, the polymeric material of the wound repair material may be prepared on a plate and formed to provide a sheet or strip that may be placed at the surface of a wound or inside a wound as necessary to prevent excess fibrosis. As an additional example, the wound repair material may be prepared in a solution or suspension with a physiologically compatible carrier medium and injected or otherwise delivered at the surface of the wound or inside a wound as necessary to prevent excess fibrosis.

As used herein, the expression “physiologically compatible carrier medium” includes any and all solvents, diluents, or other liquid vehicle, dispersion or suspension aids, surface agent agents, isotonic agents, thickening or emulsifying agents, preservatives, solid binders, lubricants, fillers and the like as are suited for the delivery of the wound repair material. Remington: The Science and Practice of Pharmacy, 20^{th} edition, A. R. Genaro et al., Part 5, Pharmaceutical Manufacturing, pp. 669-1015 (Lippincott Williams & Wilkins, Baltimore, Md./Philadelphia, Pa.) (2000) discloses various carriers used in chemical formulations and known techniques for the preparation thereof. Except insofar as any conventional pharmaceutical carrier medium is incompatible with the wound repair material of the invention, such as by producing an undesirable biological effect or otherwise interacting in an deleterious manner with any other component(s) of a formulation or material comprising the polymeric materials of the invention, its use is contemplated to be within the scope of this invention. By example, the production of liquid solutions, emulsions or suspensions or syrups one may use excipients such as water, alcohols, aqueous saline, aqueous dextrose, polyols, glycerine, lipids, phospholipids, cyclodextrins, vegetable, petroleum, animal or synthetic oils. Moreover, the wound repair material and/or polymeric materials of the invention may also contain one or more additives including, without limitation, preservatives, stabilizers, e.g., UV stabilizers, emulsifiers, sweeteners, salts to adjust the osmotic pressure, buffers, coating materials and antioxidants.

The wound repair materials of the invention may also be seeded with cells such as fibroblasts and/or keratinocytes to promote wound healing at the damaged wound tissue.

The present invention also includes methods of treating damaged wound tissue and inhibiting fibrosis in a patient in need thereof. The method may include applying a wound repair material to the wound, where the wound repair material includes a polymeric material. The polymeric material used in the methods of the invention may include PGS, and derivatives thereof, as set forth herein.

The method may include applying the wound repair material, having the polymeric material, to the wound in a pre-polymer and/or non-cross-linked state. Indeed, the polymeric material may include polymers that may be cross-linked in situ at the wound by the application of UV light, for example. Accordingly, the methods of the invention may include the step of crosslinking the polymeric material at the wound by, for example, irradiation with UV light.

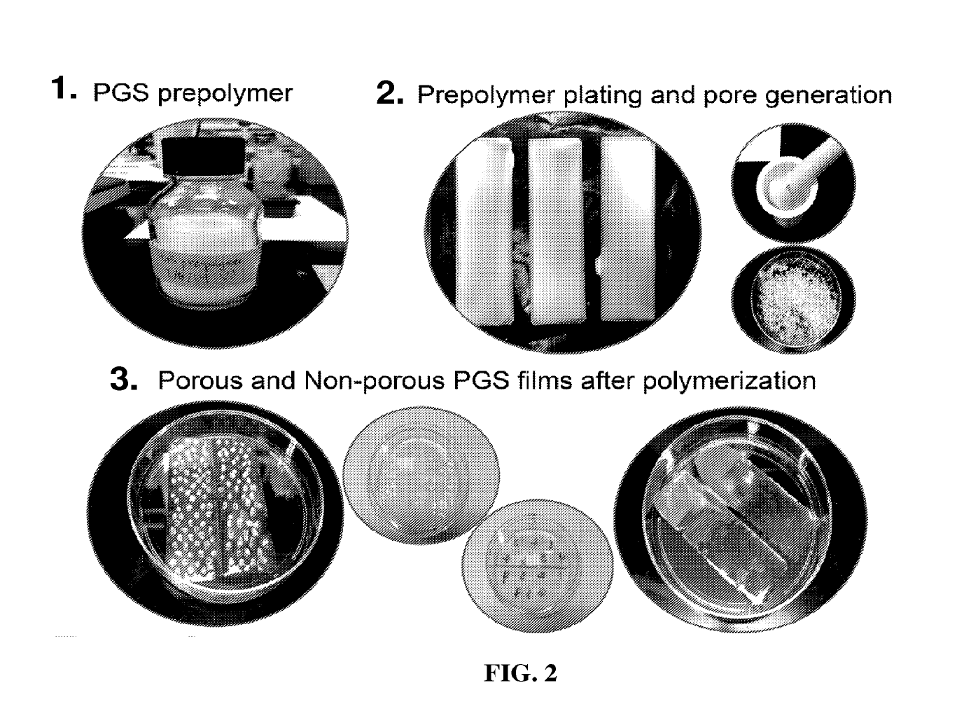

The present invention also includes methods of manufacturing wound repair materials that include polymeric materials. For example, as demonstrated in FIG. 2, both porous and non-porous wound repair materials of the invention may be prepared from a PGS prepolymer (1). The PGS prepolymer may be plated and optionally treated with salt or another satisfactory porogen that may be dissolved without interfering with the prepolymer to produce a porous wound repair material (2). The prepolymers may then be cured by treating with heat (e.g., 120° C. for 60 hours under vacuum) to produce the full length PGS polymer. Moreover, the porous wound treatment material may be placed in water or other another satisfactory solvent to dissolve the salt or porogen. Finally, the porous and non-porous PGS wound repair materials may be used after polymerization in vitro (e.g., the PGS films observed in FIG. 2 (3)) or in vivo.

As set forth herein, the materials and methods of the invention that incorporate polymeric materials, such as PGS, inhibit fibroblast proliferation and collagen production by fibroblasts and demonstrate the benefits of PGS films in regulating excess fibrosis in wounds.

Without limiting the invention in any way, the invention may be described further in the following example.

### EXAMPLE

Porous PGS films were generated to increase oxygen availability to 3T3 fibroblast cells and compared to non-porous films. Moreover, this study demonstrates that PGS may serve as a wound film cover or wound repair material and is a regulator of fibroblast growth.

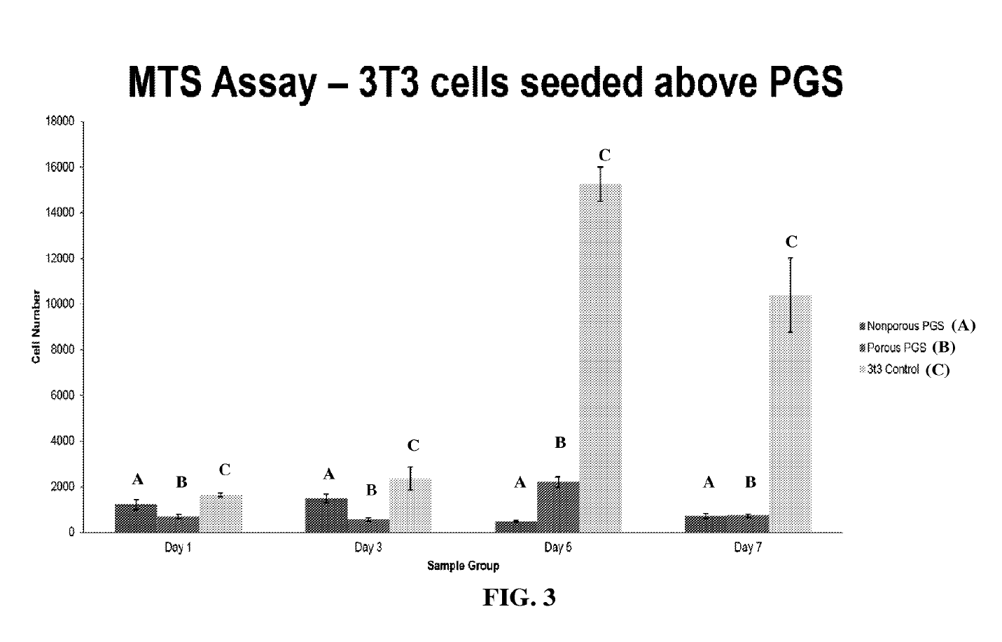

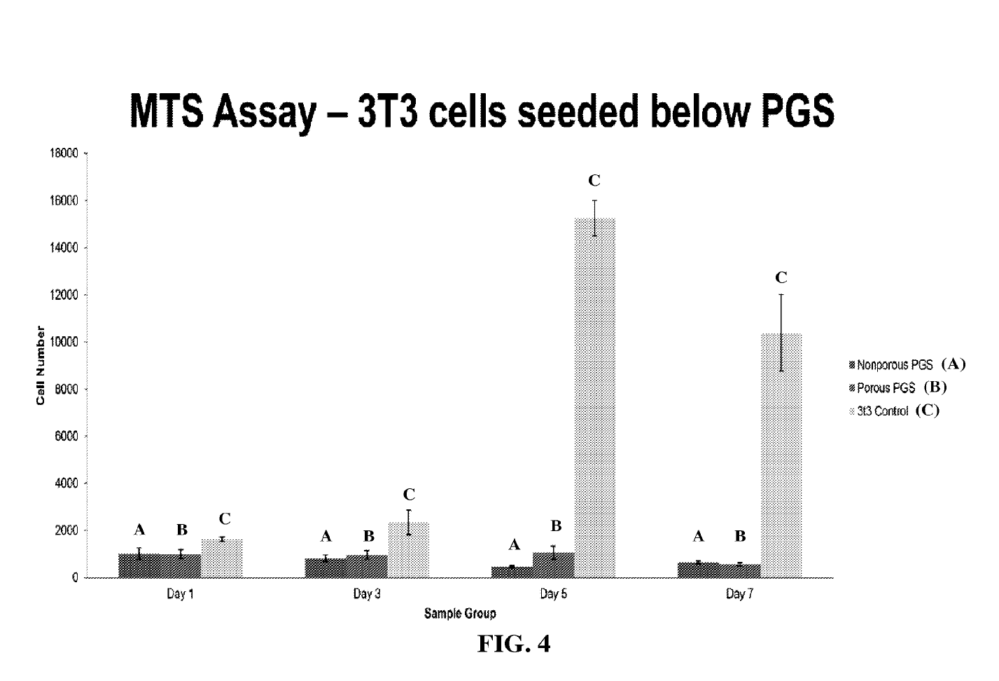

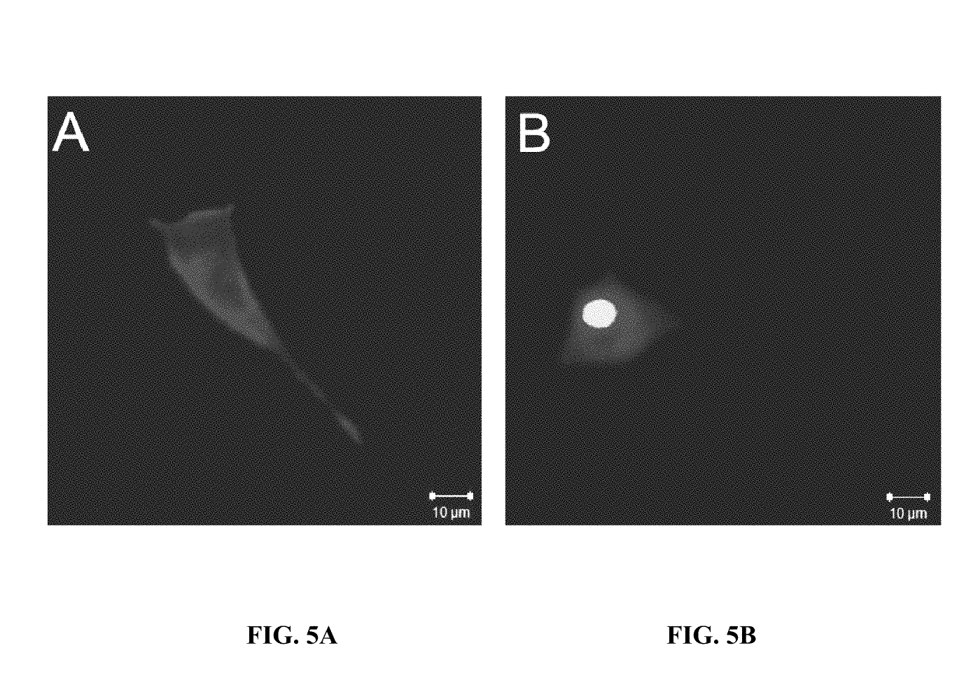

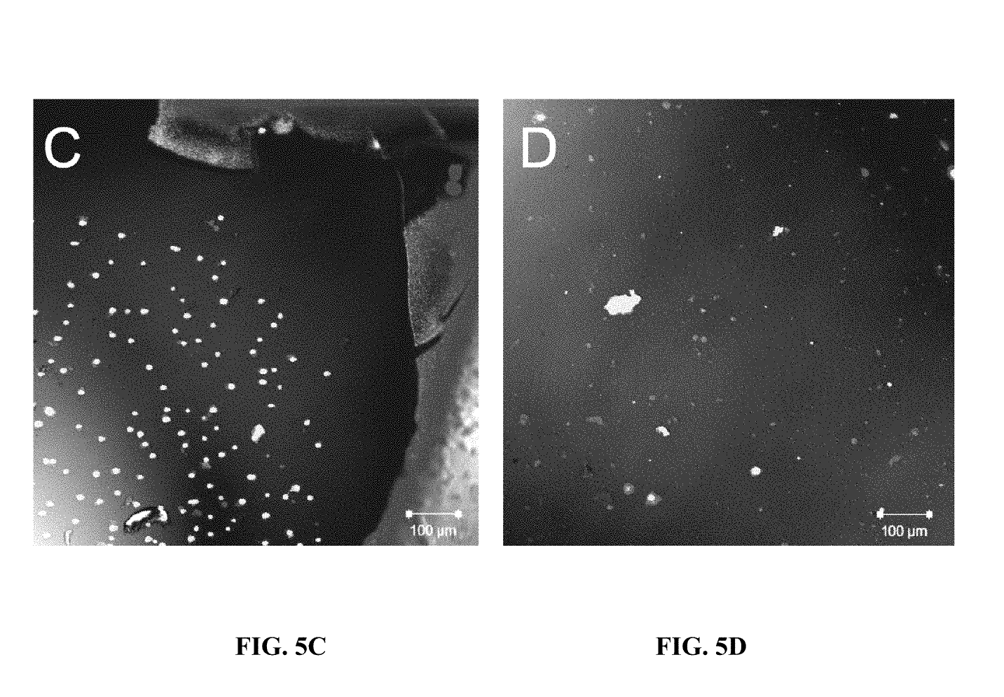

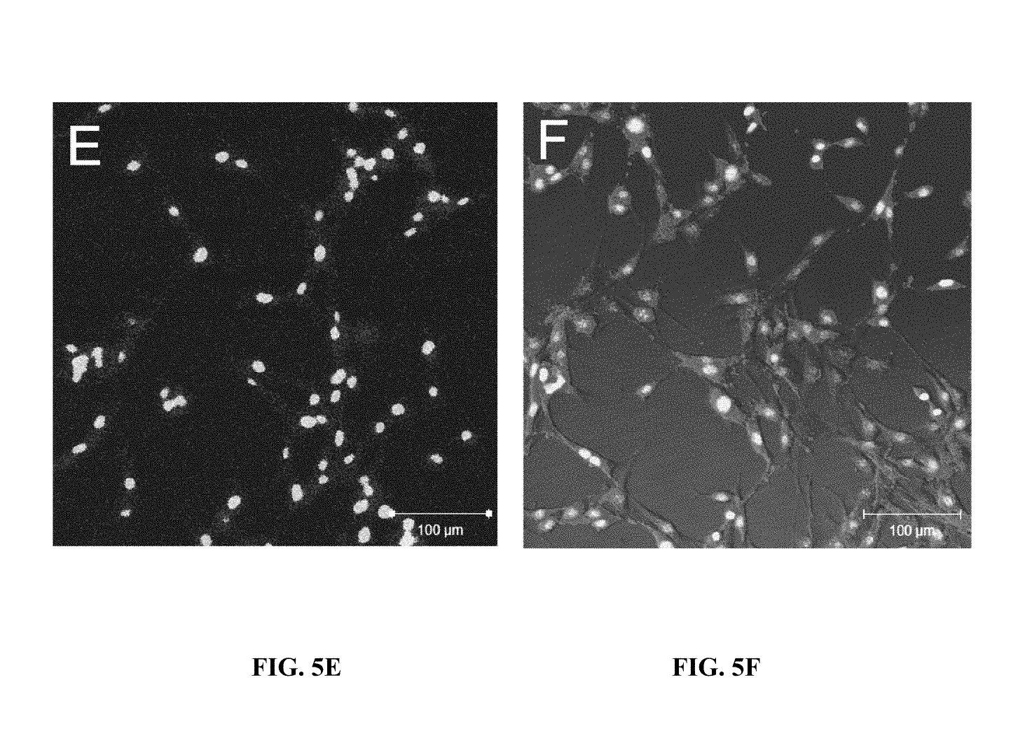

Initially, the PGS prepolymer is synthesized from glycerol and sebacic acid and plated on glass slides, with and without salt to generate pores (see, e.g., FIG. 2). The pre-polymer coated slides were cured in a 120° C. oven for 60 hours under vacuum. Following curing, the PGS-coated slides were placed in a deionized water solution for 24 hours and PGS films were removed with a razor blade. Mouse embryonic fibroblast cells (3T3 MEFs WT) (ATCC®, Manassas, Va.) were cultured in DMEM (1×) and divided into three test groups: Group I (control), Group II (porous PGS), Group III (non-porous PGS). MTS assays were performed on day 1, 3, 7 and 14 to determine cell viability in each well. Group II and III were divided into two subgroups: Subgroup A (cells plated below PGS films) (see FIG. 4); and Subgroup B (cells plated above PGS films) (see FIG. 3). Using confocal microscopy, cellular adhesion to PGS material was visualized (see FIGS. 5A-5F).

The in vitro experiments demonstrated lower cellular density in the samples plated with PGS films as compared to the control. A comparison between subgroups showed that fibroblast proliferation was negligibly affected by placement of PGS above or below the cell cultures. Presence of pores within the film improved fibroblast proliferation as compared to non-porous material.

Fibroblasts cultured in the presence of PGS films demonstrated a regulation of fibroblast proliferation as compared to no material.

A number of patent and non-patent publications may be cited herein in order to describe the state of the art to which this invention pertains. The entire disclosure of each of these publications is incorporated by reference herein.

While certain embodiments of the present invention have been described and/or exemplified above, various other embodiments will be apparent to those skilled in the art from the foregoing disclosure. The present invention is, therefore, not limited to the particular embodiments described and/or exemplified, but is capable of considerable variation and modification without departure from the scope and spirit of the appended claims.

Moreover, as used herein, the term “about” means that dimensions, sizes, formulations, parameters, shapes and other quantities and characteristics are not and need not be exact, but may be approximate and/or larger or smaller, as desired, reflecting tolerances, conversion factors, rounding off, measurement error and the like, and other factors known to those of skill in the art. In general, a dimension, size, formulation, parameter, shape or other quantity or characteristic is “about” or “approximate” whether or not expressly stated to be such. It is noted that embodiments of very different sizes, shapes and dimensions may employ the described arrangements.

Furthermore, the transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed invention. All materials and methods described herein that embody the present invention can, in alternate embodiments, be more specifically defined by any of the transitional terms “comprising,” “consisting essentially of,” and “consisting of.”

### REFERENCES

1. Wang, Y., Ameer, G. A., Sheppard, B. J., Langer, R., “A tough biodegradable elastomer,” Nature Biotechnology (2002) 20:602-606.
2. Motlagh, D., Yang, J., Lui, K. Y., Webb, A. R., Ameer, G. A., “Hemocompatibility evaluation of poly(glycerol sebacate) in vitro for vascular tissue engineering,” Biomaterials (2006) 27:4315-4324.
3. Roberts, A. B., Sporn, M. B., “Physiological actions and clinical applications of transforming growth factor-beta (TGF-beta),” Growth Factors (1993) 8:1.
4. Nijst, C. L. E., Bruggeman, J. P., Karp, J. M., Ferreira, L., Zumbuehl, A., Bettinger, C. J., and Langer, R., “Synthesis and Characterization of Photocurable Elastomers from Poly(glycerol-co-sebacate),” Biomacromolecules (2007) 8:3067-3073.

1. A wound repair material for inhibiting fibrosis in wound tissue comprising a polymeric material, wherein the polymeric material comprises a poly (glycerol sebacate) (PGS).

2. The wound repair material of claim 1, wherein the polymeric material is porous.

3. The wound repair material of claim 1, wherein the polymeric material is non-porous.

4. The wound repair material according to claim 1, wherein the polymeric material comprises poly (glycerol sebacate) acrylate (PGSA).

5. The wound repair material according to claim 1, wherein the polymeric material comprises poly (ethylene glycol) diacrylate (PEG-DA).

6. The wound repair material according to claim 1, wherein the polymeric material comprises a light activated material configured to cross-link the polymeric material upon application of UV light.

7. The wound repair material according to claim 6, wherein the light activated material is 2,2-dimethoxy-2-phenylacetophenone.

8. The wound repair material according to claim 1, wherein the polymeric material is bioabsorbable.

9. The wound repair material according to claim 1, wherein the polymeric material comprises a pre-polymer.

10. The wound repair material according to claim 1, comprising a film, a sheet, a strip, a solution, or a suspension.

11. The wound repair material according to claim 1, comprising fibroblasts, keratinocytes, or a combination thereof.

12. The wound repair material according to claim 1, comprising a physiologically compatible carrier medium.

13. A method of inhibiting fibrosis in a wound in a patient in need thereof, the method comprising applying a wound repair material to the wound, wherein the wound repair material comprises a polymeric material comprising a poly (glycerol sebacate) (PGS).

14. The method according to claim 13, wherein the wound repair material is porous.

15. The method according to claim 13, wherein the wound repair material is non-porous.

16. The method according to claim 13, wherein the polymeric material comprises poly (glycerol sebacate) acrylate (PGSA).

17. The method according to claim 13, wherein the polymeric material comprises poly (ethylene glycol) diacrylate (PEG-DA).

18. The method according to claim 13, wherein the polymeric material comprises a light activated material configured to cross-link the polymeric material upon application of UV light.

19. The method according to claim 18, wherein the light activated material is 2,2-dimethoxy-2-phenylacetophenone.

20. The method according to claim 13, wherein the wound repair material is applied as a bioabsorbable wound repair material.

21. The method according to claim 13, wherein the polymeric material comprises a pre-polymer.

22. The method according to claim 13, wherein the wound repair material is applied as a film, a sheet, a strip, a solution, or a suspension.

23. The method according to claim 13, wherein the wound comprises a non-penetrating wound, a penetrating wound, a thermal wound, a chemical wound, an electrical wound, or a combination thereof.

24. The method according to claim 13, comprising the step of cross-linking the polymeric material of the wound repair material.

25. The method according to claim 13, comprising the step of irradiating the polymeric material of the wound repair material to cross-link the polymeric material of the wound repair material.

26. The method according to claim 13, wherein the wound repair material comprises fibroblasts, keratinocytes, or a combination thereof.

27. The method according to claim 13, comprising the step of seeding the wound repair material with fibroblasts, keratinocytes, or a combination thereof.

28. The method according to claim 13, wherein the wound repair material comprises a physiologically compatible carrier medium.

29. The method according to claim 13, wherein the step of applying the wound repair material comprises delivering the wound repair material at a surface of the wound.

30. The method according to claim 13, wherein the step of applying the wound repair material comprises placing the wound repair material at a surface of the wound or inside the wound.

31. The method according to claim 13, wherein the step of applying the wound repair material comprises injecting the wound repair material inside the wound.

32-41. (canceled)

42. A repair material for inhibiting fibrosis and reducing scar formation at a wound, the repair material comprising a prepolymeric material comprising poly(glycerol sebacate) (PGS), wherein the repair material is configured to provide a porous film that increases oxygen availability at the wound.

43. The repair material of claim 42, wherein the prepolymeric material comprises poly(glycerol sebacate) acrylate (PGSA).

44. The repair material according to claim 42, comprising poly(ethylene glycol) diacrylate (PEG-DA).

45. The repair material of according to claim 42, comprising a light activated material configured to cross-link the prepolymeric material upon application of UV light.

46. The repair material according to claim 45, wherein the light activated material is 2,3-dimethoxy-2-phenylacetophenone.

47. The repair material according to claim 42, comprising fibroblasts, keratinocytes, or a combination thereof.
